(19) United States
(12) Patent Application Publication
(10) Pub. No.: US 2020/0139062 A1
(43) Pub. Date: May 7, 2020

(54) Airway Cuff Pressure Monitoring Device and Method

(71) Applicants: Southern Methodist University, Dallas, TX (US); BOARD OF REGENTS, THE UNIVERSITY OF TEXAS SYSTEM, Austin, TX (US)

(72) Inventors: Ahmet Can Sabuncu, Ashland, MA (US); Ali Beskok, Plano, TX (US); Eric Rosero, Frisco, TX (US)

(21) Appl. No.: 16/671,299

(22) Filed: Nov. 1, 2019

Related U.S. Application Data
(60) Provisional application No. 62/754,245, filed on Nov. 1, 2018.

(51) Int. Cl. A61M 16/04

(52) U.S. Cl. CPC A61M 16/044; A61M 16/0488; A61M 2016/0027; A61M 2205/8206; A61M 2205/502; A61M 16/0486

(57) ABSTRACT

The present invention includes cuff pressure monitoring device including a pressure sensor for monitoring pressure inside an airway device cuff; a T-junction coupled to the pressure sensor and capable of being placed in fluid communication with the airway device cuff and in fluid communication with an air source, wherein the T-junction is disposable; one or more electronics units coupled to the pressure sensor with a cable, wherein the cable is disposable; and a human interface coupled to the one or more electronics units, comprising one or more controls, one or more displays, or one or more sound-producing devices.

### CROSS-REFERENCE TO RELATED APPLICATIONS

This application claims priority to U.S. Provisional Application Ser. No. 62/754,245, filed Nov. 1, 2018, the entire contents of which are incorporated herein by reference.

### STATEMENT OF FEDERALLY FUNDED RESEARCH

None.

### TECHNICAL FIELD OF THE INVENTION

The present invention relates in general to the field of patient care in airway management, and more particularly, to prevention of cuff over-inflation and under-inflation in conjunction with airways.

### BACKGROUND OF THE INVENTION

Without limiting the scope of the invention, its background is described in connection with cuffs for tracheal tubes and laryngeal mask airways (LMAs).

Management of the airway during general anesthesia and mechanical ventilation in critical care patients is usually performed by tracheal intubation. Most tracheal tubes bear a cuff that, when inflated, creates a seal inside the trachea protecting the lungs from aspiration and preventing leakage of respiratory gases. The inflated cuff exerts pressure on the tracheal mucosa, the blood supply for which is provided by small terminal blood vessels. It is estimated that pressures above 25 mm Hg (or about 30 Cm H₂O) can collapse the tracheal mucosal vessels and impair perfusion. Therefore, over-inflation of the cuff of the tracheal tube can cause ischemic complications of the trachea, including sore throat, tracheal ulcers, tracheal stenosis, tracheal rupture, and tracheal-esophageal fistulas [Bennett et al. 2000, Fan et al. 2004, Hameed et al. 2008]. The recommended upper limit of tracheal cuff pressure is 30 Cm H₂O to avoid tracheal complications and the lower limit is 20 Cm H₂O to avoid pulmonary aspiration. Several studies indicate that during general anesthesia and other tracheal intubation scenarios, tracheal cuff over-inflation occurs in more than 50% of patients [Parwani et al. 2007, Pisano et al. 2017, Sengupta et al. 2004, Stewart et al. 2003]. This is due both to unawareness among anesthesia, intensive care, and emergency care providers and lack of portable devices to check the cuff pressure. A study conducted by the inventors at Clements University Hospital (Rosero et al. 2018) demonstrated that the incidence of cuff over-inflation in non-cardiac surgery was 89% and that in about 46% of the patients the cuff pressure was higher than 100 Cm H₂O. Over-inflation occurred despite the type of anesthesia provider; i.e. anesthesiologist, certified registered nurse anesthetist, or resident. A survey among the anesthesia providers and Clements Hospital indicated that the lack of a portable manometer was the principal barrier to adequate inflation of the tracheal cuff (Rosero et al., 2016). Another way to manage the airway during anesthesia in selected patients and procedures is the use of a laryngeal mask airway (LMA). LMAs are supra-glottic airway devices that partially seal the airway by inflation of a cuff above the larynx. Over-inflation of the LMA cuffs is also very frequent and is associated with many upper airway complications.

Patent application WO 2015/136337 A1, by Vega et al., is said to disclose a device for measuring pressures in the cuff of endotracheal tubes, said device comprising: a casing; a liquid crystal display that displays the pressures detected; a securing means for the connecting cannula; a cannula connected to the pilot balloon of the tube cuff where the pressure sensor is located; an electronic processing card; a micro-controller having a 12-bit analogue-digital converter; a signal conditioning circuit; a high-intensity LED serving as a visual alert and a buzzer serving as an acoustic alert; a button for displaying different pressures; and a supply battery. The device is also provided with a low-noise amplifier disposed between the pressure sensor and the microcontroller.

Therefore, there is a need for a low-cost, lightweight, easily portable, easy-to-use, reusable pressure-monitoring device with disposable sensing and connection elements to monitor the inflation of tracheal and LMA cuffs during anesthesia and outside the operating room.

### SUMMARY OF THE INVENTION

In some embodiments of the disclosure, a cuff pressure monitoring device includes a pressure sensor for monitoring pressure inside an airway device cuff; a T-junction coupled to the pressure sensor and capable of being placed in fluid communication with the airway device cuff and in fluid communication with an air source, wherein the T-junction is disposable; one or more electronics units coupled to the pressure sensor with a cable, wherein the cable is disposable; and a human interface coupled to the one or more electronics units, including one or more controls, one or more displays, or one or more sound-producing devices. In one aspect, the device further includes a power source or a connection to a power source. In another aspect, the power source comprises a rechargeable battery. In another aspect, the device further includes an amplifier coupled to the pressure sensor and the one or more electronics units to amplify a signal from the pressure sensor. In another aspect, the device further includes a controllable pressure release valve in fluid communication with the cuff and capable of being operated automatically or manually. In another aspect, the controllable pressure release valve is operated by the controller or is programmable and not operated by the one or more electronics units. In another aspect, the one or more electronics units configured to perform analog-to-digital conversion, processing of data from the pressure sensor, generation of output to the human interface, or processing of input from the human interface. In another aspect, the pressure sensor is capable of monitoring the pressure in the airway device cuff intermittently or continuously. In another aspect, the airway device cuff operates with an airway device comprising a tracheal tube or a laryngeal mask airway. In another aspect, the device is portable. In another aspect, the one or more electronics units are reusable for multiple patients. In another aspect, the device is capable of turning off automatically. In another aspect, the cuff pressure monitoring device monitors for maintenance of an airway device cuff pressure within a specified operating range, and wherein the specified operating range is set or adjusted by programming the one or more electronics units or changing the pressure sensor. In another aspect, the air source is a pressurizing device.

In some embodiments of the disclosure, a kit includes cuff pressure monitoring device including: a pressure sensor for monitoring pressure inside an airway device cuff; a T-junction coupled to the pressure sensor, and capable of being placed in fluid communication with the airway device cuff and in fluid communication with an air source, wherein the T-junction is disposable; one or more electronics units coupled to the pressure sensor with a cable, wherein the cable is disposable; and a human interface coupled to the one or more electronics units, including one or more controls, one or more displays, or one or more sound-producing devices. In one aspect, the cuff pressure monitoring device further includes a power source or a connection to a power source. In another aspect, the power source comprises a rechargeable battery. In another aspect, the cuff pressure monitoring device further includes an amplifier coupled to the pressure sensor and the one or more electronics units to amplify a signal from the pressure sensor. In another aspect, the cuff pressure monitoring device further includes a controllable pressure release valve in fluid communication with the cuff and capable of being operated automatically or manually. In another aspect, the controllable pressure release valve is operated by the controller or is programmable and not operated by the one or more electronics units. In another aspect, the one or more electronics units are configured to perform analog-to-digital conversion, processing of data from the pressure sensor, generation of output to the human interface, or processing of input from the human interface. In another aspect, the pressure sensor is capable of monitoring the pressure in the airway device cuff intermittently or continuously. In another aspect, the airway device cuff operates with an airway device comprising a tracheal tube or a laryngeal mask airway. In another aspect, the device is portable. In another aspect, the one or more electronics units are reusable for multiple patients. In another aspect, the device is capable of turning off automatically. In another aspect, the cuff pressure monitoring device monitors for maintenance of an airway device cuff pressure within a specified operating range, and wherein the specified operating range is set or adjusted by programming the one or more electronics units or changing the pressure sensor. In another aspect, the air source is a pressurizing device.

In some embodiments of the disclosure, a method of determining an airway device cuff pressure includes providing a patient requiring installment of an airway device; installing an airway device equipped with a cuff; coupling a cuff pressure monitoring device to the cuff, wherein the cuff pressure monitoring device includes: a pressure sensor; a T-junction coupled to the pressure sensor and capable of being placed in fluid communication with an airway device cuff and in fluid communication with an air source, wherein the T-junction is disposable; one or more electronics units coupled to the pressure sensor with a cable, wherein the cable is disposable; and a human interface coupled to the one or more electronics units, comprising one or more controls, one or more displays, or one or more sound-producing devices; inflating the cuff with the air source; displaying an airway device cuff pressure with the human interface; and producing an audio or visual warning with the human interface when the airway device cuff pressure is outside a preselected operating range of pressure. In one aspect, the cuff pressure monitoring device further comprises a power source or a connection to a power source. In another aspect, the cuff pressure monitoring device further comprises an amplifier coupled to the pressure sensor and the one or more electronics units to amplify a signal from the pressure sensor. In another aspect, the cuff pressure monitoring device further comprises a controllable pressure release valve in fluid communication with the cuff and capable of being operated automatically or manually. In another aspect, the controllable pressure release valve is operated by the controller or is programmable and not operated by the one or more electronics units. In another aspect, the one or more electronics units are configured to perform analog-to-digital conversion, processing of data from the pressure sensor, generation of output to the human interface, or processing of input from the human interface.

### BRIEF DESCRIPTION OF THE DRAWINGS

For a more complete understanding of the features and advantages of the present invention, reference is now made to the detailed description of the invention along with the accompanying figures and in which:

FIG. 1 depicts a block diagram of an embodiment of the present invention.

FIG. 2 depicts a flowchart of a method embodiment of the present invention.

FIGS. 3A and 3B depict portions of an experimental embodiment of the present invention.

FIGS. 4A-4F illustrate six calibration points of the experimental embodiment of FIGS. 3A and 3B of the present invention.

FIG. 5 shows a pressure-voltage calibration curve for the experimental embodiment of the present invention of FIGS. 3A, 3B, and FIGS. 4A-4F.

FIG. 6 depicts an exemplary prototype of the present invention.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

The present invention includes embodiments that feature a lightweight, portable pressure monitoring device that consists of a pressure sensor, which may be a micro electromechanical system (MEMS), coupled to one or more electronics units, a human interface, and a disposable T-junction, which connects the pressure sensor to the cuff of the airway device, e.g., a tracheal tube or an LMA. These embodiments can be used to measure intermittently or continuously the pressure inside the cuff. Anesthesia providers and other personnel can use these embodiments to keep the cuff pressure within a safe range.

Endotracheal intubation requires a tube to be inserted from the mouth of a patient down into the trachea to initiate artificial respiration. This procedure is common during general anesthesia and in other emergency conditions. The endotracheal tube (or “tracheal tube”) is a plastic tube that is connected to a gas source on one end and having an orifice on the other end for the delivery of gas into the patient's lung. The tube also bears an inflatable cuff that provides a seal against leakage of respiratory gases and pulmonary aspiration of bodily fluids. In a regular practice, the cuff is pressurized using a syringe, resulting in increased pressure in tracheal mucosa. As discussed elsewhere herein, over-inflation is common.

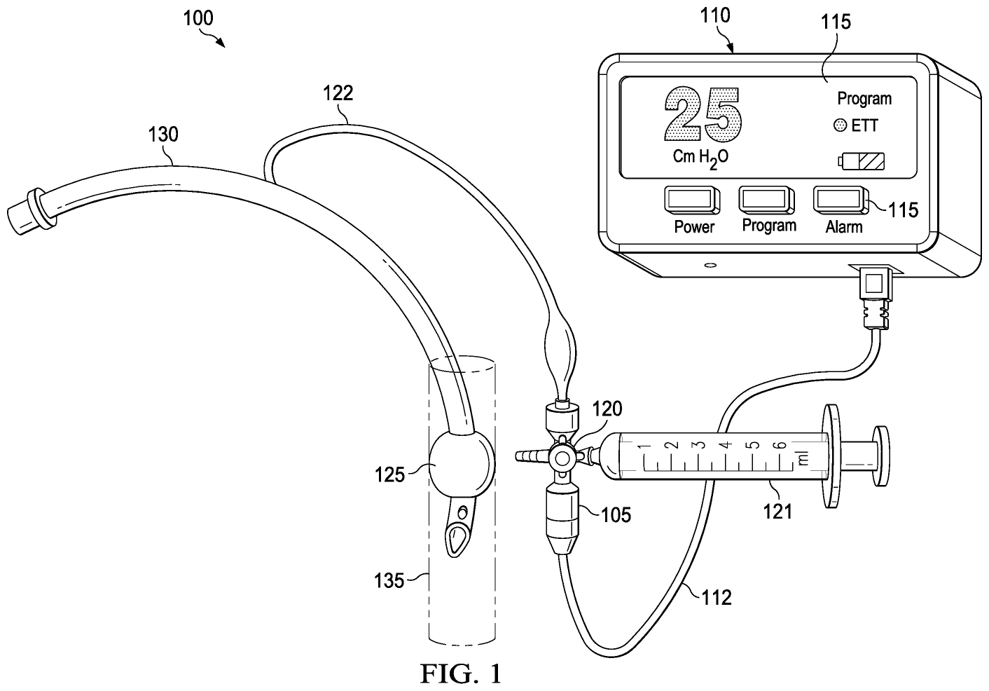

FIG. 1 depicts a block diagram of an embodiment of the present invention. Cuff pressure-monitoring device 100 includes a pressure sensor 105, which is coupled to one or more electronic units 110. The pressure sensor 105 may be, for example, a low-cost microelectromechanical system (MEMS). The pressure sensor 105 is coupled to the one or more electronic units 110 with a cable 112, e.g., a USB cable or a low-cost custom-built cable system, which is disposable after use to prevent cross-contamination among patients. In some embodiments, a receptacle to which the cable couples at the one or more electronic units 110 is waterproof, and lets no blood or other fluid from entering the one or more electronic units 110. The other construction of the one or more electronic units 110 is made using a material selected for easy cleaning such as stainless steel or polycarbonate.

The one or more electronic units 110, in turn, are connected to one or more human interfaces 115 that includes, for example, one or more displays or sound-producing devices (e.g., audible alarms) for data and one or more controls for human operation. The human interface may include, e.g., one or more liquid crystal displays (LCDs) capable of one or more modes of data display such as numerical or graphical display, one or more light-emitting diode (LED) bar graphs, and a buzzer that sounds when the pressure is out of limits, and the cuff pressure may be shown in a number of units, e.g., kPa or cm H₂O. In FIG. 1, the one or more electronic units 110 and a human interface 115 are shown as separate items coupled together, but one or more human interfaces 115 may be parts of a single integral unit with the one or more electronic units 110. Embodiments of the present invention can be programmed for use with different airway devices, e.g., a tracheal tube or an LMA. The desired operating pressure range can be set or adjusted by programming the one or more electronic units 110 or by changing the pressure sensor 105. Embodiments of the present invention may turn off automatically.

In FIG. 1, The pressure sensor 105 is coupled to one of the branches of a T-junction 120 with an adapter (not shown), while another branch of the T-junction 120 is capable of being placed in fluid communication to an air supply, which may be a pressurizing device such as a syringe 121, and a third branch of the T-junction 120 is capable of being placed in fluid communication via, e.g., a tube 122, with the cuff 125 of an airway device 130, shown here inside a patient's trachea 135. The airway device 130 may be, e.g., a tracheal tube or an LMA. In one embodiment, the T-junction 120 has a three-way valve to expose the pressure sensor 105 to, and to disconnect the pressure sensor 105 from, the pressurized air in the cuff 125. The T-junction is disposable to prevent cross-contamination among patients, so embodiments maybe used for more than one patient.

Embodiments of the invention include an inbuilt constant power supply or a coupling to a constant power supply, such a battery that may be rechargeable, for the pressure sensor 105, an instrumentation amplifier, e.g., a low-power amplifier, to amplify the signal from the pressure sensor 105, and/or a signal conditioner to condition the signal from the pressure sensor 105. Embodiments of the invention also include a controllable pressure release valve in fluid communication with the cuff to keep the pressure within the desired limits. Such a valve may be operated automatically or manually. It may be coupled to and controlled by the one or more electronic units 110 using information from the pressure sensor 105, or it may be programmable and not operated by the one or more electronic units. The valve may be an integral part of the pressure sensor and the T-junction, or it may be coupled to the gas tubing already described by a second T-junction.

Embodiments of the present invention are low-cost, lightweight, easily portable, easy-to-use, and reusable for multiple patients. Embodiments of the present invention can be used for other applications that require intermittent or continuous measurements of pressures inside a body cavity, including esophageal pressure measurements, bladder pressure measurement, or intra-abdominal pressure monitoring.

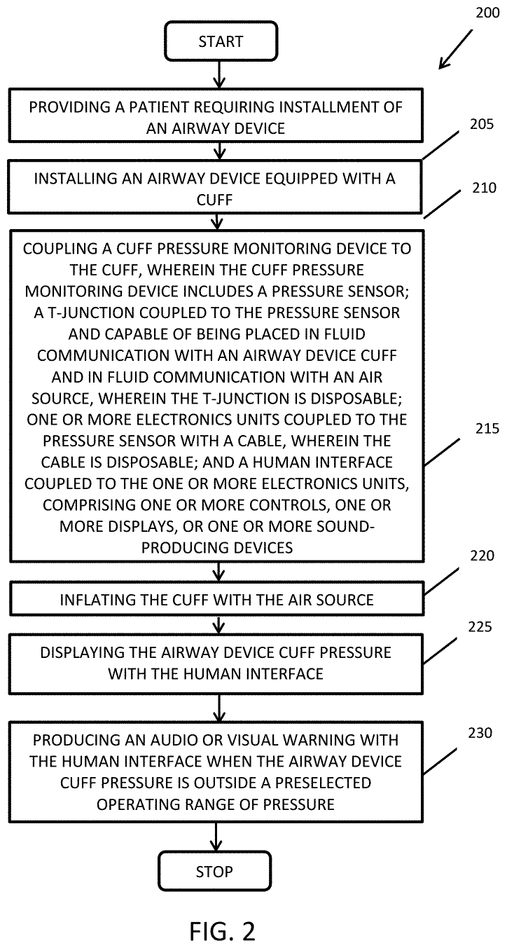

FIG. 2 illustrates a flowchart for a method embodiment of the present invention. Method 200 is a method of determining an airway device cuff pressure. Method 200 with block 205, providing a patient requiring installment of an airway device. Block 210 continues with installing an airway device equipped with a cuff. Block 215 includes: coupling a cuff pressure monitoring device to the cuff, wherein the cuff pressure monitoring cuff includes a pressure sensor; a T-junction coupled to the pressure sensor and capable of being placed in fluid communication with an airway device cuff and in fluid communication with an air source, wherein the T-junction is disposable; one or more electronics units coupled to the pressure sensor with a cable, wherein the cable is disposable; and a human interface coupled to the one or more electronics units, comprising one or more controls, one or more displays, or one or more sound-producing devices. The cuff is inflated with the air source in block 220. Block 225 includes displaying an airway device cuff pressure with the human interface. Block 230 includes producing an audio and/or visual warning with the human interface when the airway device cuff pressure is outside a preselected operating range of pressure.

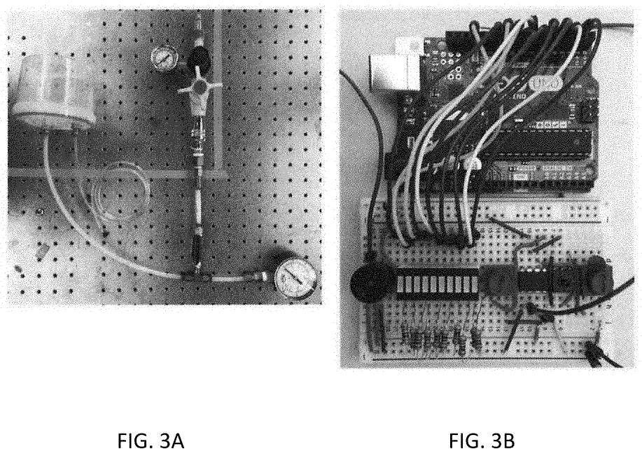

FIG. 3A depicts a portion of an experimental embodiment of the present invention, including a pressure transducer, a mechanical pressure gauge for calibration, and a testing chamber. FIG. 3B depicts another portion of the experimental embodiment depicted in FIG. 3A, including an Arduino UNO (an open-source microcontroller board), a breadboard and electronic components.

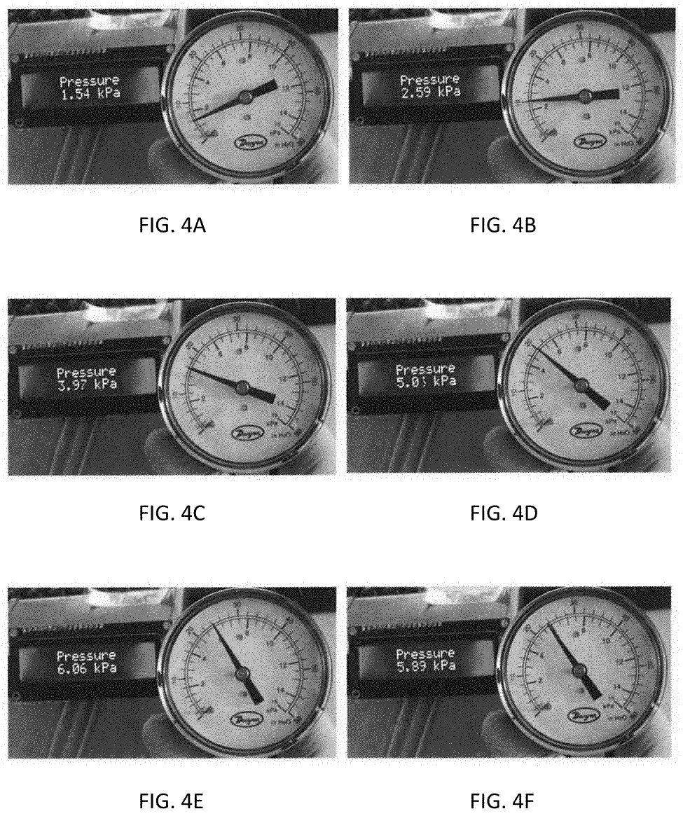

FIGS. 4A-4F illustrate six calibration points of the experimental embodiment of FIGS. 3A and 3B of the present invention using a mechanical pressure gauge. The calibration information is recorded and programmed into the Arduino UNO.

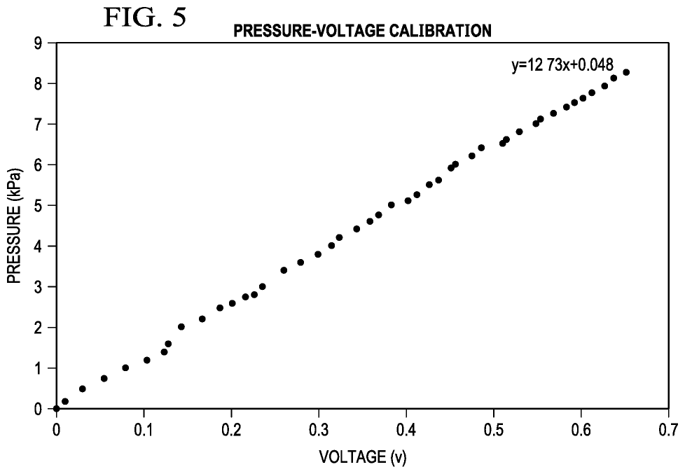

FIG. 5 shows a pressure-voltage calibration curve for the experimental embodiment of the present invention of FIGS. 3A, 3B, and FIGS. 4A-4F.

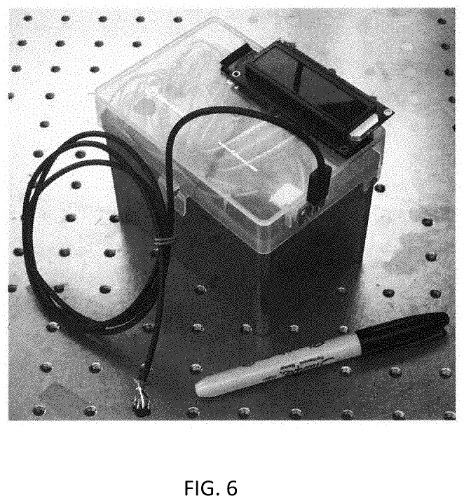

FIG. 6 illustrates an exemplary prototype of an embodiment of the present invention next to a pen to provide scale.

One skilled in the art of designing or using airway devices will recognize that the present invention, including but not limited to the cuff pressure monitoring device 100 and the method 200 of determining an airway device cuff pressure, is a low-cost, lightweight, easily portable, easy-to-use, reusable pressure-monitoring device with a disposable sensing element to control the inflation of tracheal and LMA cuffs during anesthesia and outside the operating room.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke paragraph 6 of 35 U.S.C. § 112 as it exists on the date of filing hereof unless the words “means for” or “step for” are explicitly used in the particular claim.

### REFERENCES

Bennett M H, Isert P R, Cumming R G. Postoperative sore throat and hoarseness following tracheal intubation using air or saline to inflate the cuff—a randomized controlled trial. Anaesth Intensive Care 2000; 28:408-13.

Fan C M, Ko P C, Tsai K C et al. Tracheal rupture complicating emergent endotracheal intubation. Am J Emerg Med 2004; 22:289-93.

Hameed A A, Mohamed H, Al-Mansoori M. Acquired tracheoesophageal fistula due to high intracuff pressure. Ann Thorac Med 2008; 3:23-5.

Parwani V, Hoffman R J, Russell A et al. Practicing paramedics cannot generate or estimate safe endotracheal tube cuff pressure using standard techniques. Prehosp Emerg Care 2007; 11:307-11.

Pisano A. Pitfalls From Physics: Why We Can't “Feel” the Tube Cuff Pressure With Our Fingers. Anesth Analg 2017.

Sengupta P, Sessler D I, Maglinger P et al. Endotracheal tube cuff pressure in three hospitals, and the volume required to produce an appropriate cuff pressure. BMC Anesthesiol 2004; 4:8.

Stewart S L, Secrest J A, Norwood B R, Zachary R. A comparison of endotracheal tube cuff pressures using estimation techniques and direct intracuff measurement. AANA J 2003; 71:443-7.

Rosero E B, Michael M, Hairr J, Hansen K. Preventing Over-inflation of the Tracheal Tube Cuff During General Anesthesia. University of Texas System, Shared Visions Meeting. San Antonio, Tex., Apr. 21, 2016.

Rosero E B, Ozayar E, Eslava-Schmalbach J, Minhajuddin A, Joshi G P. Effects of Increasing Airway Pressures on the Pressure of the Endotracheal Tube Cuff During Pelvic Laparoscopic Surgery. Anesth Analg. 2018 July; 127(1):120-125.

What is claimed is:

1. A cuff pressure monitoring device comprising:
a pressure sensor for monitoring pressure inside an airway device cuff;
a T-junction coupled to the pressure sensor and capable of being placed in fluid communication with the airway device cuff and in fluid communication with an air source, wherein the T-junction is disposable;
one or more electronics units coupled to the pressure sensor with a cable, wherein the cable is disposable; and
a human interface coupled to the one or more electronics units, comprising one or more controls, one or more displays, or one or more sound-producing devices.

2. The device of claim 1, further comprising a power source or a connection to a power source.

3. The device of claim 2, wherein the power source comprises a rechargeable battery.

4. The device of claim 1, further comprising an amplifier coupled to the pressure sensor and the one or more electronics units to amplify a signal from the pressure sensor.

5. The device of claim 1, further comprising a controllable pressure release valve in fluid communication with the cuff and capable of being operated automatically or manually.

6. The device of claim 5, wherein the controllable pressure release valve is operated by the one or more electronics units or is programmable and not operated by the one or more electronics units.

7. The device of claim 1, wherein the one or more electronics units are configured to perform analog-to-digital conversion, processing of data from the pressure sensor, generation of output to the human interface, or processing of input from the human interface.

8. The device of claim 1, wherein the pressure sensor is capable of monitoring the pressure in the airway device cuff intermittently or continuously.

9. The device of claim 1, wherein the airway device cuff operates with an airway device comprising a tracheal tube or a laryngeal mask airway.

10. The device of claim 1, wherein the device is portable.

11. The device of claim 1, wherein the one or more electronics units are reusable for multiple patients.

12. The device of claim 1, wherein the device is capable of turning off automatically.

13. The device of claim 1, wherein the cuff pressure monitoring device monitors for maintenance of an airway device cuff pressure within a specified operating range, and wherein the specified operating range is set or adjusted by programming the one or more electronics units or changing the pressure sensor.

14. The device of claim 1, wherein the air source is a pressurizing device.

15. A kit comprising:
cuff pressure monitoring device comprising:
a pressure sensor for monitoring pressure inside an airway device cuff;
a T-junction coupled to the pressure sensor, and capable of being placed in fluid communication with the airway device cuff and in fluid communication with an air source, wherein the T-junction is disposable;
one or more electronics units coupled to the pressure sensor with a cable, wherein the cable is disposable; and
a human interface coupled to the one or more electronics units, comprising one or more controls, one or more displays, or one or more sound-producing devices.

16. The kit of claim 15, wherein the cuff pressure monitoring device further comprises a power source or a connection to a power source.

17. The kit of claim 16, wherein the power source comprises a rechargeable battery.

18. The kit of claim 15, wherein the cuff pressure monitoring device further comprises an amplifier coupled to the pressure sensor and the one or more electronics units to amplify a signal from the pressure sensor.

19. The kit of claim 15, wherein the cuff pressure monitoring device further comprises a controllable pressure release valve in fluid communication with the cuff and capable of being operated automatically or manually.

20. The kit of claim 19, wherein the controllable pressure release valve is operated by the one or more electronics units or is programmable and not operated by the one or more electronics units.

21. The kit of claim 15, wherein the one or more electronics units are configured to perform analog-to-digital conversion, processing of data from the pressure sensor, generation of output to the human interface, or processing of input from the human interface.

22. The kit of claim 15, wherein the pressure sensor is capable of monitoring the pressure inside the airway device cuff intermittently or continuously.

23. The kit of claim 15, wherein the airway device cuff operates with an airway device comprising a tracheal tube or a laryngeal mask airway.

24. The kit of claim 15, wherein the device is portable.

25. The kit of claim 15, wherein the one or more electronics units reusable for multiple patients.

26. The kit of claim 15, wherein the device is capable of turning off automatically.

27. The kit of claim 15, wherein the cuff pressure monitoring device monitors for maintenance of an airway device cuff pressure within a specified operating range, and wherein the specified operating range is set or adjusted by programming the one or more electronics units or changing the pressure sensor.

28. The kit of claim 15, wherein the air source is a pressurizing device.

29. A method of determining an airway device cuff pressure comprising:
providing a patient requiring installment of an airway device;
installing an airway device equipped with a cuff;
coupling a cuff pressure monitoring device to the cuff, wherein the cuff pressure monitoring device comprises:
a pressure sensor;
a T-junction coupled to the pressure sensor and capable of being placed in fluid communication with an airway device cuff and in fluid communication with an air source, wherein the T-junction is disposable;
one or more electronics units coupled to the pressure sensor with a cable, wherein the cable is disposable; and
a human interface coupled to the one or more electronics units, comprising one or more controls, one or more displays, or one or more sound-producing devices;
inflating the cuff with the air source;
displaying an airway device cuff pressure with the human interface; and
producing an audio or visual warning with the human interface when the airway device cuff pressure is outside a preselected operating range of pressure.

30. The method of claim 29, wherein the cuff pressure monitoring device further comprises a power source or a connection to a power source.

31. The method of claim 29, wherein the cuff pressure monitoring device further comprises an amplifier coupled to the pressure sensor and the one or more electronics units to amplify a signal from the pressure sensor.

32. The method of claim 29, wherein the cuff pressure monitoring device further comprises a controllable pressure release valve in fluid communication with the cuff and capable of being operated automatically or manually.

33. The method of claim 32, wherein the controllable pressure release valve is operated by the one or more electronics units or is programmable and not operated by the one or more electronics units.

34. The method of claim 29, wherein the one or more electronics units are configured to perform analog-to-digital conversion, processing of data from the pressure sensor, generation of output to the human interface, or processing of input from the human interface.
